(19) United States
(12) Patent Application Publication
(10) Pub. No.: US 2005/0214898 A1
(43) Pub. Date: Sep. 29, 2005

(54) Methods of carotenoid production in bacteria

(76) Inventors: Paul S. Bernstein, Salt Lake City, UT (US); Prakash Bhosale, Salt Lake City, UT (US)

Correspondence Address: TRASK BRITT, P.O. BOX 2550, SALT LAKE CITY, UT, 84110, US

(21) Appl. No.: 11/091,662

(22) Filed: Mar. 28, 2005

Related U.S. Application Data
(60) Provisional application No. 60/557,743, filed on Mar. 29, 2004.

(51) Int. Cl. C12P 23/00; C12N 1/20

(52) U.S. Cl. 435/67; 435/252.3

(57) ABSTRACT

Methods of supplementing a growth medium with means for enhancing the production of a carotenoid in a bacterium are disclosed. In various embodiments, the growth medium is supplemented with a salt or a TCA cycle intermediate to enhance the production of the carotenoid. Bacterial cell cultures including the means for enhancing the production of the carotenoid in the bacterium are also disclosed.

### CROSS-REFERENCE TO RELATED APPLICATION

This application claims the benefit of U.S. Provisional Application No. 60/557,743, filed Mar. 29, 2004, the contents of the entirety of which are incorporated herein by this reference.

### STATEMENT REGARDING FEDERALLY SPONSORED RESEARCH OR DEVELOPMENT

Work described herein was supported in part by National Institute of Health Grant No. EY-11600. The United States Government may have certain rights in the invention.

### TECHNICAL FIELD

The present invention relates generally to the field of biotechnology, and more particularly, to methods for producing carotenoids in bacteria.

### BACKGROUND

Carotenoids are antioxidant micronutrients reported to protect against many medical illnesses. Among the carotenoids, β-carotene is the most recognized and widely known carotenoid. In the past decade, several clinical trials and epidemiological studies have indicated β-carotene's utility against several types of cancers (Holick et al., 2002; Nishino et al., 2002). Along with its medicinal usages, β-carotene is used in the cosmetics, food and feed industries due to its colorant and antioxidant properties (Gordon and Bauernfeind, 1982; Edge et al., 1997).

Researchers and biotechnologists have successfully commercialized microbial sources of β-carotene. Alga Dunaliella and fungi Blakeslea trispora are two prominent β-carotene producers reported in the literature (Nelis and DeLeenheer, 1991; Johnson and Schroeder, 1996). There are also reports on mutation programs carried using yeast Xanthophyllomyces dendrorhous and Rhodotorula glutinis for the hyperproduction of 1-carotene (Girard et al., 1994; An 1996; Bhosale and Gadre, 2001).

Efforts to use non-carotenogenic bacteria for carotenoid production by use of recombinant DNA technology are yet to reach the commercial stage (Misawa and Shimada H, 1997; Lee and Schmidt-Dannert, 2002). It appears that no reports on potential carotenogenic bacteria as a β-carotene producer exist. Among carotenogenic bacterial sources, Synechococcus and Flavobacterium sp. are reported to produce β-carotene as a minor product (Nelis and DeLeenheer, 1991).

Flavobacterium sp. has been widely studied for production of the xanthophyll carotenoid zeaxanthin (Pasamontes et al., 1997; Alcantara and Sanchez, 1999; Masetto et al., 2000; Bhosale et al., 2003), especially since zeaxanthin may be protective against age related macular degeneration (Moeller et al., 2000). β-Carotene and β-cryptoxanthin are known to act as precursors in the biochemical pathway of zeaxanthin production and, thus, appreciable levels of these carotenoids (˜5-10%) were observed during initial growth phases of Flavobacterium sp. (McDermott et al., 1973; Bhosale et al., 2003). Hydroxylation of β-carotene and β-cryptoxanthin ultimately leads to the accumulation of zeaxanthin.

There are several inhibitors which inhibit the hydroxylation process reported in the literature, however the effect was also found to be inhibitory to growth (McDermott et al., 1973). Inhibitors of carotenoid biosynthetic pathway have been previously used to produce unconventional carotenoids from conventional microbes (Ducrey Sanpietro and Kula, 1998). The inhibitors can be used in the minimum concentrations for inter carotenoid transformation without affecting growth. Quantitative optimization of inhibitors can be done using statistical approaches.

Biosynthetic studies show that zeaxanthin is synthesized from 1-carotene by hydroxylation of C(3) and C(3′) of the 1 ring via monohydroxy intermediate 1 cryptoxanthin, a process that requires molecular oxygen in a mixed-function oxidase reaction. Logically, inhibition of hydroxylase activity should lead to the accumulation of β-carotene. There are few inhibitors such as, for example, CaCl₂.6H₂O and solid alkaloid salts reported in the literature which leads to the accumulation of β-carotene (McDermott et al. 1973). However, the use of the inhibitors is limited to biochemical studies only, as most of the inhibitors also severely inhibit growth.

“Response surface methodology” is a statistical method which can be employed in microbial biotechnology to study the effect of media components that play a decisive role in the process. It can be coupled with a “method of steepest ascent” to optimize the quantitative values of media supplements. It has also been used by many researchers in assessing the role of media components in carotenoid production (Ramirez et al., 2001; Vazquez and Martin, 1998).

Several microbial and non-microbial sources have been reported and exploited for their potential commercialization. Among microbial sources, the production of β-carotene by an alga Dunaliella sp. is a well-developed technology (Ben-Amotz, 1998; Ben-Amotz, 1999). However, the production of carotenoids by slow growing microalgae, particularly Dunaliella sp., requires maintenance of stress conditions such as salt concentration and intense light. In addition, the microalgae have a peculiar requirement of CO₂ and oxygen during day and night cycles of growth, respectively.

A fungus, Blakeslea trispora, is also capable of producing a high amount of β-carotene. The detailed studies on production parameters have been reported (Kim et al., 1996; Seon-Won et al., 1997). However, the production of carotenoids by B. trispora is dependent upon sexual mating of two compatible strains during fermentation. In addition, the growth of B. trispora in fermentors becomes viscous and needs a considerable energy input to keep it aerobic and well mixed.

The production of β-carotene by a mutant of yeast Xanthophyllomyces dendrorhous and Rhodotorula glutinis was also reported (Girard et al., 1994; Bhosale and Gadre 2001), but their commercial potential is still under investigation. There are very few bacterial sources available in the research reports. Johnson and Schroeder, 1996, have listed a few of these 1-carotene producing bacteria For instance, Flavobacterium multivorum ATCC 55238 is reported in the literature for the production of xanthophyll carotenoids such as zeaxanthin (Masetto et al., 2001; Bhosale et al., 2003). However, most of β-carotene producing bacteria produce 1-carotene as a minor product.

Previously, supplementation of urea in a growth medium is indicated to increase volumetric yield of carotenoid in Phaffia rhodozyma (Fontana 1996) and at a cellular level in Rhodotorula glutinis (Bhosale and Gadre 2001), but the alteration of a biochemical pathway of the bacterium in the presence of urea is not known. In contrast, sodium carbonate is mainly used as nutrient source by a few alkophilic microorganisms, and is not known for enhancing carotenoid production. Urea and sodium carbonate are likely to have a detrimental effect on hyrdoxylase activity, thus, leading to major inhibition of zeaxanthin formation.

### SUMMARY OF THE INVENTION

It has been surprisingly found that a bacterium growing in a culture medium supplemented with a salt or a Citric Acid Cycle (i.e., the Krebs cycle), hereinafter a “TCA intermediate,” is enabled to enhance the production of carotenoids by the bacterium. The invention thus includes the use of a supplemented culture medium to grow or ferment the bacterium such that the production of carotenoids by the bacterium is enhanced. As used herein, the term “enhanced,” in addition to its normal English definition, means that an amount of carotenoids normally produced by the bacterium, i.e., produced by growing the bacterium in a culture medium that is not supplemented, will be increased or augmented. The carotenoids may be isolated from the culture medium and/or the bacterium, optionally purified. Thus, in another embodiment, the carotenoids may be used in pharmaceuticals, neutriceuticals, food stuffs, cosmetics, and in the animal feed industry by admixing the carotenoid with the pharmaceuticals, nutriceuticals, food stuffs, cosmetics, or animal feed.

In one embodiment, a bacterium such as, for example, Flavobacterium multivorum was discovered to be a producer of a carotenoid such as, for example, β-carotene by manipulation of the culture such as by supplementing the culture media with a salt such as, for example, urea, sodium carbonate and combinations thereof. (See, Table 5 of FIG. 11, indicating that F. multivorum grown using the methods of the instant invention produces more carotenoids than other bacteria). Thus, it will be advantageous to the microbial fermentation industry to exploit the potential of this organism as a β-carotene source since bacteria have the advantage over algae and fungus of having a faster growth rate. For instance, Table 5 of FIG. 11 shows comparative levels of β-carotene from some of the major β-carotene producing bacteria From Table 5 of FIG. 11, it is evident that F. multivorum produces the highest amount of β-carotene among the carotenogenic and non carotenogenic recombinant bacteria.

In another embodiment, the effect of supplementation of a growth medium with several salts on carotenoid production from the bacterium F. multivorum ATCC 55238 was discovered. Selected salts were optimized using a sequential approach of response surface methodology (RSM) and a method of steepest ascent. Profiles of production of carotenoids were studied in the optimized medium.

In one embodiment, β-carotene was produced as a major carotenoid (≧70%) in a growth medium supplemented with urea and sodium carbonate. The carotenoids were analyzed by HPLC (see, FIG. 3) and confirmed by matching absorption spectra with the authentic carotenoid. Urea and sodium carbonate supported enhanced β-carotene production from Flavobacterium.

In another embodiment, the interactive effect of two salt supplements was studied using response surface methodology experiments to maximize the β-carotene level without drastically affecting the growth performance of the organism. The sequential optimization approach led to a substantial increase in β-carotene proportion and content (see, FIG. 2), which is significant on an industrial scale. Thus, the instant invention implicates the use of fast growing bacteria as a source of carotenoids.

In another embodiment, the effect of intermediates of the TCA cycle on carotenoid production from the bacterium F. multivorum ATCC 55238 was discovered. It was determined that isocitric acid, malic acid and α-ketoglutarate had a maximum stimulatory effect on the volumetric production of the carotenoid zeaxanthin.

In another embodiment, the carotenoids produced by the bacterium using the methods of the instant invention may be administered as a pharmaceutical, neutraceutical or food stuff composition to a subject. Such a composition includes the carotenoids and a pharmaceutically acceptable carrier such as, for example, lactose, cellulose, or equivalent, or contained within a pharmaceutical dosage such as a capsule or tablet. In a further embodiment, the carotenoid is admixed with an animal feed for administration to an animal.

In a further embodiment, the carotenoids are produced on a large scale in a fermentor or in a culture medium. The fermentor may include an enhanced bacterial growth medium comprising at least one of glucose, yeast extract, and peptone. The fermentor also includes a means for enhancing the production of a carotenoid in a bacterium and a bacterium capable of producing carotenoid in an enhanced proportion as compared to the bacterium growing in a medium lacking the means for enhancing the production of a carotenoid. In one embodiment, the means for enhancing the production of a carotenoid comprises calcium chloride, lithium chloride, magnesium chloride, magnesium sulfate, sodium carbonate, sodium chloride, sodium phosphate, ammonium chloride, ammonium phosphate, urea, isocitric acid, malic acid, α-ketoglutarate, citric acid or combinations of any thereof

### BRIEF DESCRIPTION OF THE DRAWINGS

FIG. 1 is a response surface plot for the interactive effect of urea and sodium phosphate concentration on β-carotene production levels.

FIGS. 2A and 2B represent step-wise improvements in the β-carotene content (A) and proportion (B) in the approach described herein.

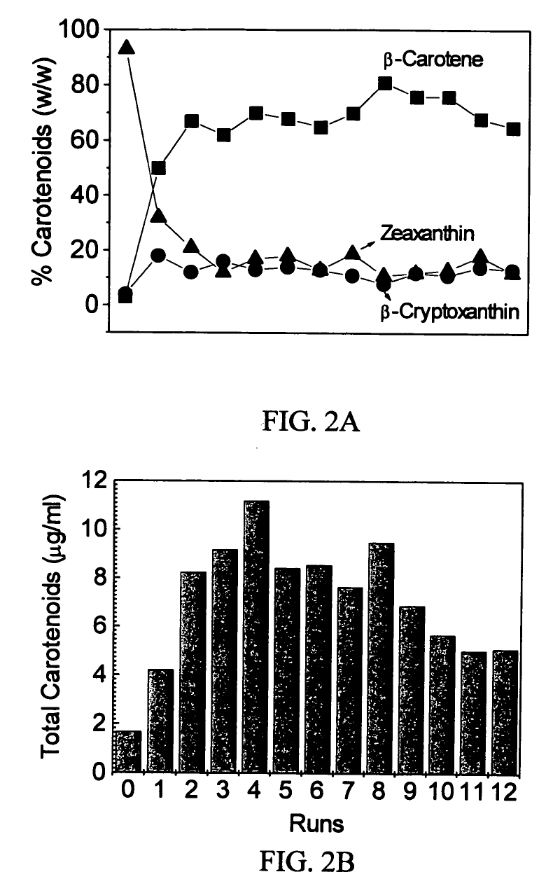

FIG. 3 are normalized HPLC chromatograms showing relative levels of β-carotene (β-C), β-cryptoxanthin (β-Cryp), and zeaxanthin (ZX) levels at 48 h in the optimized medium (line) and basal medium (dashed).

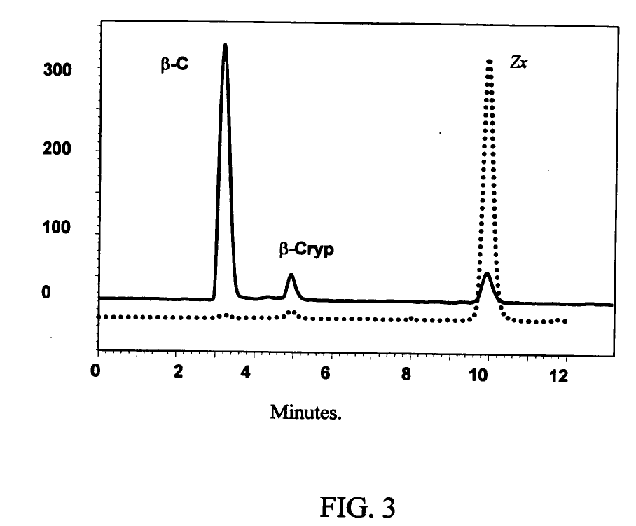

FIG. 4 is a growth pattern (OD at 500 nm) and production profile (μg/ml) of β-carotene, β-cryptoxanthin and zeaxanthin by F. multivorum ATCC 55238 grown in basal (open symbols) and optimized medium (closed symbols).

FIG. 5 illustrates the effects of TCA intermediates on zeaxanthin production in a bacterium.

FIG. 6 compares the production of zeaxanthin in an optimal media and a control media

FIG. 7 illustrates Table 1.

FIG. 8 illustrates Table 2.

FIG. 9 illustrates Table 3.

FIG. 10 illustrates Table 4.

FIG. 11 illustrates Table 5.

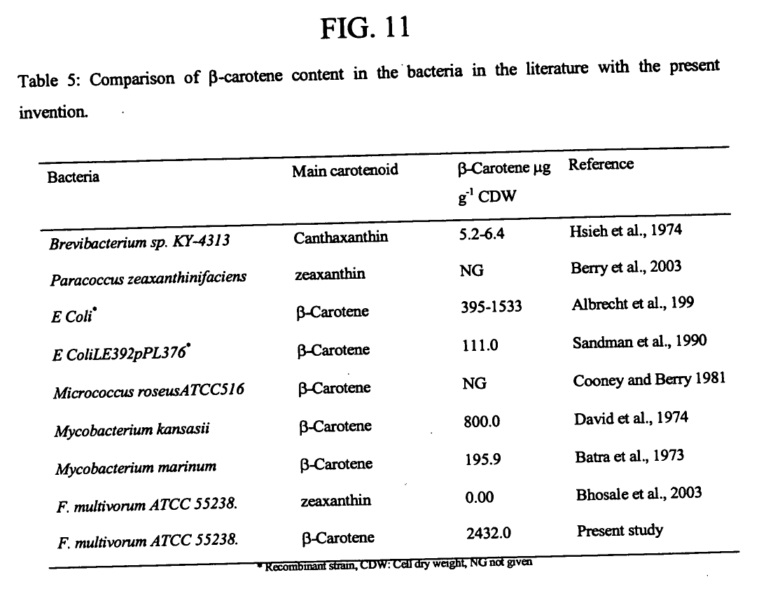

### BEST MODE OF THE INVENTION

In various embodiments, the effect of supplementation of salts or TCA cycle intermediates in growth medium on the growth, total carotenoid production, and proportion of β-carotene, β-cryptoxanthin, and zeaxanthin by F. multivorum is disclosed. Several salts had a positive effect on the total carotenoid production and the observed improvement was about 1.5-2 fold. In one embodiment, urea and sodium carbonate had a positive effect on β-carotene production. The effect was found to be independent of incubation time and β-carotene represented 70% (w/w) of the total carotenoid content. The cumulative effect of urea and sodium carbonate was determined using response surface methodology. In one embodiment, optimum medium was found to contain between about 400 and 407 mg/100 ml of urea and sodium carbonate, respectively. The maximum β-carotene level was 7.85 μg/ml, which represented 80% (w/w) of the total carotenoid. Optimization resulted in 77 and 88 fold improvement in the volumetric and cellular β-carotene level, respectively, accompanied by a simultaneous decrease in the zeaxanthin level as compared to the control medium. The carotenoid production profile in the optimized medium indicated that β-carotene was produced maximally during late exponential at 0.41 μg/ml/h.

The invention is further explained with the aid of the following illustrative Examples.

### EXAMPLES

Materials and Methods.

Inoculum. A 5% (v/v) inoculum of F. multivorum ATCC 55238 in the logarithmic phase (14 h), grown in the basal medium described herein was used throughout the studies (A₅₀₀ nm 0.8).

Reagents and chemicals. Media ingredients and β-carotene were obtained from Sigma Chemical. HPLC grade methylene chloride, methanol and hexane were obtained from Fisher Scientific. Synthetic zeaxanthin and β-cryptoxanthin were obtained from Hoffmann-La Roche (Basel, Switzerland). The mineral salts of Table 1 were obtained from Amresco, Solon (Ohio).

Bacterial strains and growth (culture) medium. F. multivorum ATCC 55238 was obtained from American Type Culture Collection (ATCC, Manassas, Va.). Cultures of F. multivorum were maintained on YM agar containing (g/l) glucose 35, malt extract 30, yeast extract 20, peptone 10, MgSO₄.7H₂O 0.2, and agar 25, at pH 6.0. The basal liquid growth medium for shake flask studies contained, in g/l, glucose 25, yeast extract 10 and peptone 10 at a pH 7.0.

### Example I

Pigment extraction from bacterial cells. One ml of culture broth was centrifuged at 5000 rpm for 10 min at 4° C. The supernatant was discarded and media components of the cell pellet were washed away by repeated suspension in sterile distilled water and centrifugation. The cell mass was subjected to sonication using a sonic dismembrator (Fisher Scientific, Model number F60) in the presence of 1 ml cold, oxygen-free methanol containing 0.01% butylated hydroxy toluene (BHT) (w/v) for 30 seconds (output power 5). Methanol extraction was repeated for complete extraction of carotenoids. The sonicated sample was centrifuged to remove the white cell pellet. The supernatant contains extracted carotenoids.

### Example II

High performance liquid chromatography (HPLC). The pigments in methanol were dried by vacuum evaporation in a Speedvac Plus (SC 110, Savant) and re-dissolved in one ml of HPLC mobile phase (hexane: dichloromethane: methanol: N,N′-di-isopropylethylamine (80:19.2:0.7:0.1 v/v)). HPLC separation was carried out at a flow rate of 1.0 ml min⁻¹ on a cyano column (Microsorb 25 cm length×4.6 mm id, Rainin Instrument Co. Woburn, Mass., U.S.A.). The column was maintained at room temperature and the HPLC detector was operated at 450 nm. Peak identities were confirmed by photodiode-array spectra and by coelution with authentic standards as necessary.

### Example II

Cell growth measurement. Cell growth was monitored by measurement of turbidity at 500 mm with a UV-visible spectrophotometer (Smart Spec™ 3000, BioRad). Samples were suitably diluted (so as to have absorbance of between 0.2 to 0.8) with double distilled water. The absorbance was measured immediately at 500 nm. For cell dry weight (CDW) estimation, the 10 ml sample was centrifuged at 10,000 g for 10 minutes and washed twice with double distilled water by suspension and centrifugation. The supernatant was discarded and the cell pellet was taken to constant weight in an oven at 80° C.

### Example 1V

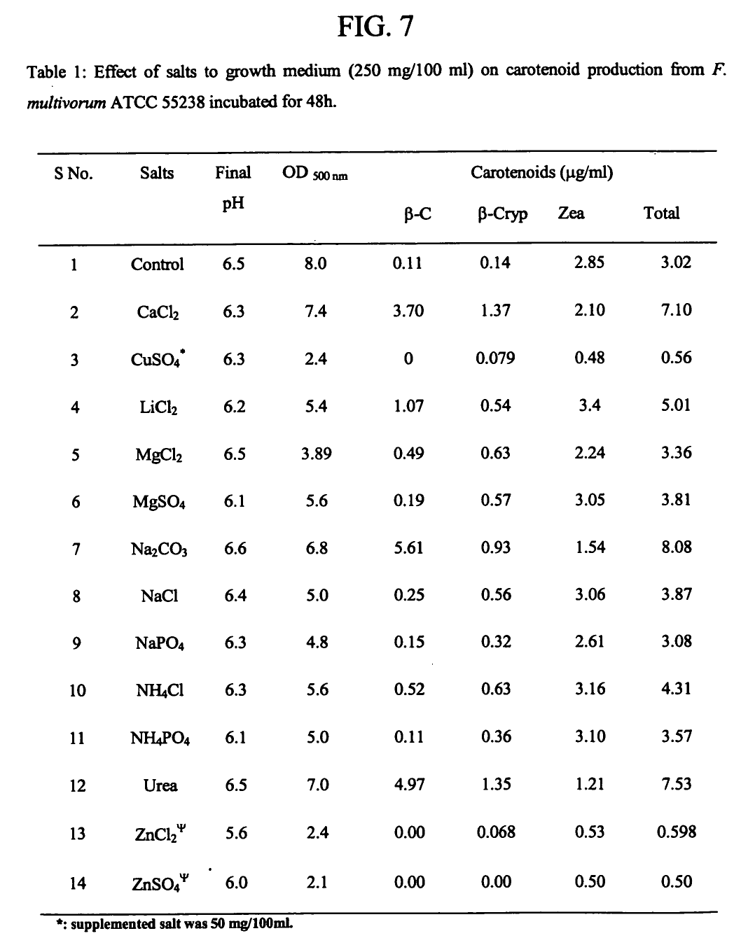

Effect of salt supplementation. F. multivorum was studied for growth and carotenoid production in the liquid basal medium supplemented with the salts listed in Table 1 (see, FIG. 7) (250 mg/100 ml) to evaluate the effect of mineral salts. The F. multivorum cultures were grown in quadruplicate in shake flask cultures at 30° C. on a rotary shaker rotates at 250 rpm.

### Example V

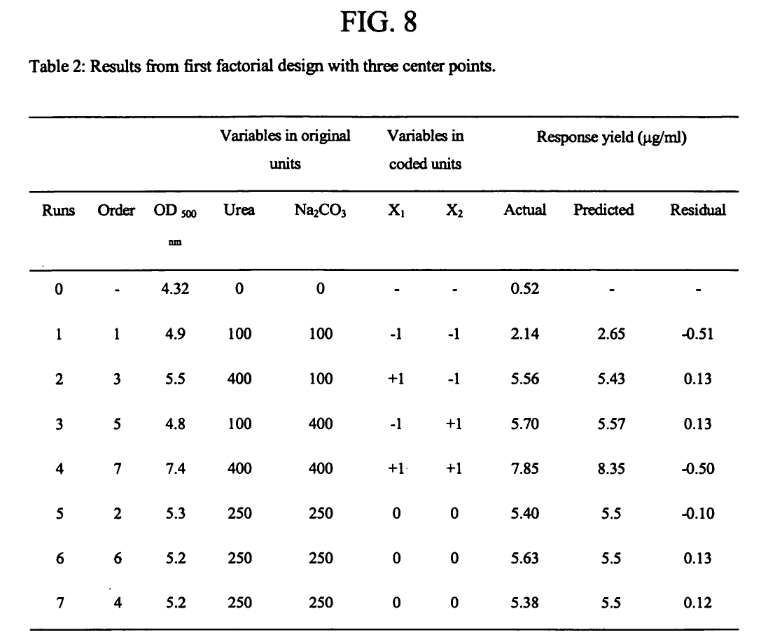

Response surface methodology. After initial discoveries, urea (U) and sodium carbonate (SC), both of which promoted 1-carotene production, were selected as independent variables in a central composite design for optimization using response surface methodology. A 2² first order factorial design with three center points (250 mg1100 ml each) was performed in random order as listed in Table 2 (see, FIG. 8) (Box et al., 1978; Bayne and Rubin 1986; Davies, 1993; Montgomery, 1997). All variables were taken at central coded value considered as zero.

The minimum and maximum ranges of variables investigated with respect to their values in actual (100 & 400 mg/100 ml) and coded form (−1 & +1) was determined. The levels of the variables in coded units were X₁ and X₂ for urea and sodium carbonate, respectively. Upon completion of studies, the average maximum volumetric production of β-carotene (μg/ml) was used to assess the response. It allowed efficient fitting and checking of the first-degree polynomial model which was used to estimate the predicted response Yₚ. Yₚ=β₀+β₁X₁+β₂X₂ (Equation 1).

This model was chosen on the ground that the predominant local characteristics of the surface were its gradients and that the local surface could be represented by the planar model (Equation 1) having slope β₁ in the X₁, and slope β₂ in the X₂ direction. β₀ represented the intercept in the equation. Further optimization using a method of steepest ascent using suitable step size was as previously described (Bayne and Rubin 1986; Montgomery, 1997).

### Example VI

Growth and carotenoid production in optimized medium. Growth and carotenoid production by F. multivorum were determined using optimized medium in shake flasks (in quadruplicate) at 30° C., on a rotary shaker at 250 rpm. Samples were periodically removed and analyzed for cell growth and carotenoid production.

### Example VII

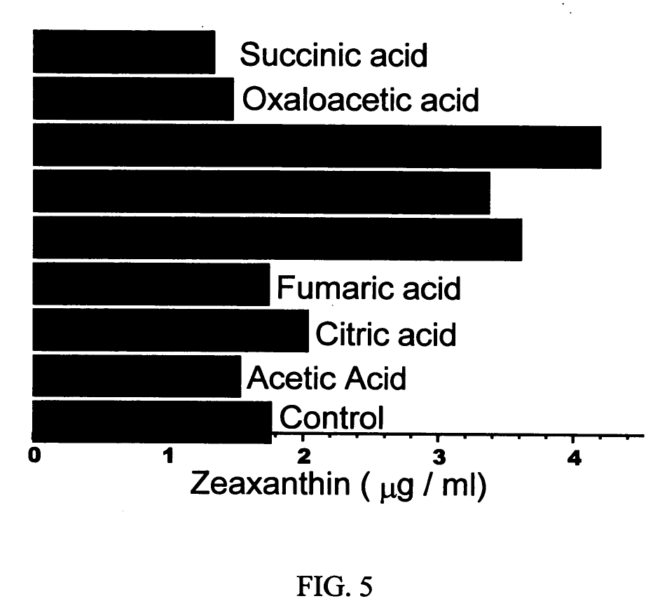

Media optimization for zeaxanthin production. It was discovered that supplementation of growth media with intermediates of the TCA cycle had a significant stimulatory effect on zeaxanthin production in F. multivorum. Isocitric acid, malic acid and α-ketoglutarate were found to have a maximum stimulatory effect on the volumetric production of zeaxanthin (μg/ml) (see, FIG. 5). Quantitative levels of isocitric acid, malic acid and α-ketoglutarate were optimized using two level three variable factorial design and a method of steepest ascents.

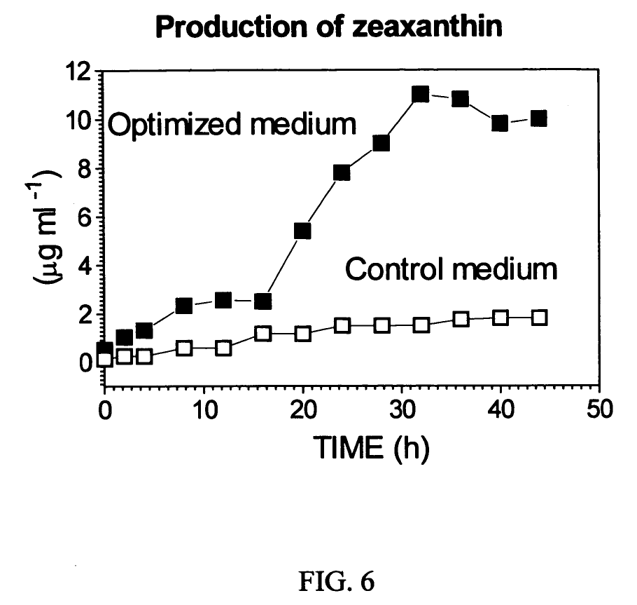

In the control medium, zeaxanthin, β-cryptoxanthin, and β-carotene contents were 1.77±0.09, 0.24±0.03, and 0.076±0.01 μG/ml, respectively. Cellular accumulation (μg/g) of zeaxanthin was not affected much upon supplementation with TCA intermediates. In most cases, zeaxanthin was found to be 90 ±2% (w/w) of the total carotenoid content, with the remainder as β-cryptoxanthin (8 ±2%), β-carotene (1.5 ±0.25), and lutein (0.5 ±0.1%). The highest level of zeaxanthin (10.65±0.63 μg ml-1) was discovered with malic acid, isocitric acid and α-ketoglutarate having levels of 6.02, 6.20 and 0.02 mM, respectively. An optimization approach displayed six-fold improvement in the zeaxanthin production as compared to the control (see, FIG. 6).

Results

Effect of supplementation of salts. Urea, sodium carbonate and calcium chloride had a maximum stimulatory effect on the volumetric production of β-carotene (βg/ml, Table 1, see FIG. 7) and represented 66, 69, 52% (w/w) of the total carotenoid content, respectively, while exhibiting a minor inhibition on growth. Lithium chloride supported β-carotene production, but was observed to be to be time dependent as the maximum β-carotene level (44%, w/w) was observed at 24 h of growth phase which eventually fell to 21% (w/w) at the end of fermentation (48 h). Zeaxanthin was the major carotenoid in the presence of ammonium and magnesium salts and both had an effect on carotenoid production pattern and final growth of the bacteria Zinc and copper salts had a negative effect on growth even at low concentrations.

Optimization by response surface methodology. The effect of Urea (U) and sodium carbonate (SC) on β-carotene production was studied using response surface methodology. Taking 250 mg/100 ml as a starting point, concentrations of urea and sodium carbonate were varied from 100 to 400 mg/100 ml as shown in Table 2 (see FIG. 8). Seven studies were sequentially performed (runs 1-7). Table 2 (see FIG. 8) summarizes the central composite design along with the experimental and predicted responses in each individual experiment.

The least squares estimates of β₁ were calculated using the method described by Box et al. (1978). Least squares estimates of intercept β₀ is the average of the seven observations and was calculated to be 5.5. Statistical analysis indicated that sodium carbonate (β₂=1.46) was a little more influential than urea (β₁=1.39), individually. The fitted equation Yₚ=5.5±1.39x₁+1.46x₂ (Equation 2) was obtained. Experimental values displayed excellent correspondence to the predicted values.

Montgomery (1996) proposed that 22 should allow the researcher to 1) obtain an estimate or error, 2) check for interactions in the model, and 3) check for quadratic effects. All three proposed calculations are essential to explore the adequacy of the first order model.

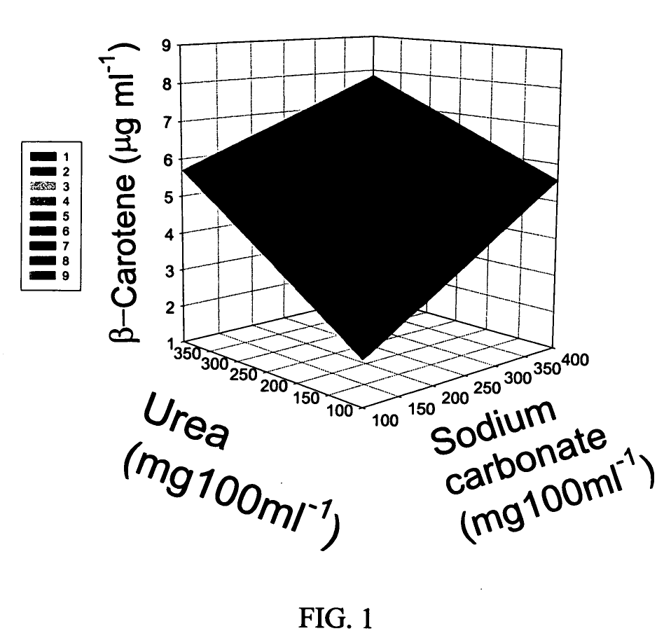

An estimate of error was calculated using the replicates at the center in the design to be 0.52. The interaction between the variables could be measured by the coefficient β₁₂ of an added cross product term X₁X₂ in the model. The coefficient was observed to be −0.3175. The interactive effect between these two factors represented in a response surface plot (see, FIG. 1) and a maximum estimated response was obtained at the combinations of higher coded values (+1) of both urea and sodium carbonate. The three dimensional response surface curves were plotted to understand the interaction of the urea and sodium carbonate.

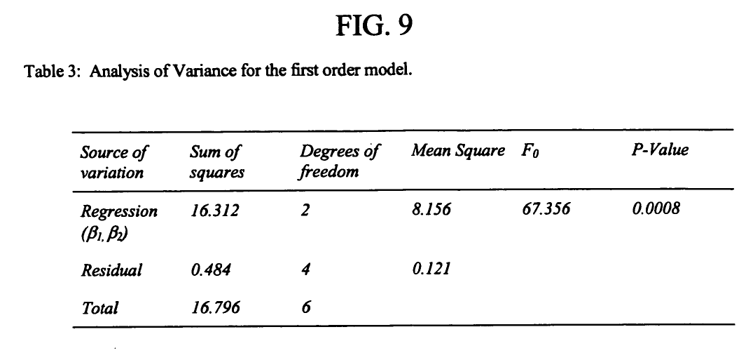

The adequacy of the straight-line model was confirmed by applying the check for quadratic (curvature). The lack of a fit statistic obtained by comparing the sum of square pure quadratic to an estimate of error resulted in a very low value (0.008) indicating that there is no quadratic effect. The analysis of the variance for this model is summarized in Table 3 (FIG. 9). The interaction and curvature checks were not significant, whereas the F test for the overall regression is significant. The standard error of β₁ and β₂ is 0.36 (√σ²/4). Both regression coefficients β₁ and β₂ are large relative to their standard errors. Hence, the model was observed to be well suited for optimization.

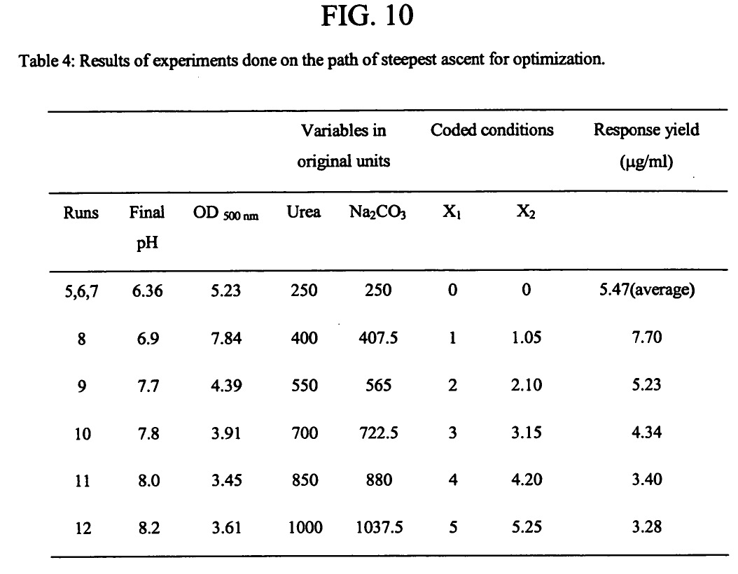

In order to look for optimum value, points on a path of steepest ascent were selected by moving 1.46 units in x₂ for every 1.39 units moved in x₁ as shown in Table 4 (FIG. 10) (runs 8-12). The optimum combination was observed to contain 400 and 407.5 mg/100 ml of urea and sodium carbonate, respectively, which resulted in 7.7 μg/ml P carotene. Further increases in the quantities lead to a decrease in β-carotene content and growth. This may be because of unfavorable growth conditions due to an increase in pH of the growth medium (Table 4, see FIG. 10). Optionally, a second factorial could be designed using the best combination in the steepest ascent experiment as central data points. In this case however, the optimum combination was very close to a fourth run (Table 2, see FIG. 8) in the first factorial experiment.

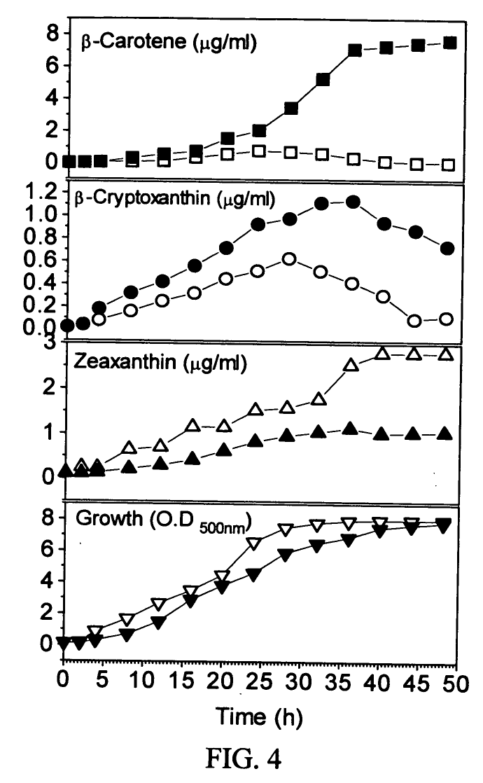

Carotenoid Production profile. Growth and carotenoid production profiles of F. multivorum ATCC 55238 were studied in salt supplemented optimized medium and compared with the basal medium lacking salts (see, FIG. 4). The maximum specific growth rate (μₘₐₓ) and maximum biomass achieved with optimum medium was 0.24/h and 3.21 g/l, respectively, as compared to 0.36/h and 3.28 g/l obtained in the basal medium. Supplementation of the medium with urea and sodium carbonate resulted in a slower growth rate of bacteria, but eventually recovered to achieve almost a similar cell mass.

In the optimized medium, the production profile indicated that β-carotene was the major carotenoid produced throughout the run (see, FIG. 4). A maximum production rate for β-carotene was observed to be between 24-36 h (0.41 μg/ml/h), respectively, as compared to the maximum obtained in the control medium (0.09 μg/ml/h) obtained between 20-24 h. Zeaxanthin, on the other hand, displayed a decrease in the maximum production rate in the optimized medium (0.05 μg/ml/h) as compared to the control basal medium (0.13 μg/ml/h). μ-Cryptoxanthin was produced at almost a similar rate in both control and optimized medium.

In the basal medium, a maximum total volumetric carotenoid was observed to be 3.02±0.6 μg/ml, while the cellular level was observed to be 921 μg/g, representing β-carotene, β-cryptoxanthin, and zeaxanthin in the proportion of 3:4:93(%, w/w). In the optimized medium, total volumetric and cellular carotenoid content was observed to be 9.49±0.6 μg/ml and 2965 μg/g, respectively, and β-carotene, β-cryptoxanthi and zeaxanthin were in the proportion of 82:7:11 (%, w/w). Thus, through sequential experimental optimization, β-carotene displayed 77 and 88 fold improvement in the volumetric production and cellular accumulation, respectively.

The exemplary embodiments described herein are not meant to limit the scope of the present invention. The present invention may be carried out using embodiments different from those specifically described herein. Therefore, the scope of the present invention is not limited by the exemplary embodiments, but is defined by the appended claims.

### REFERENCES

Alcantara, S., Sanchez, S. 1999. Influence of carbon and nitrogen sources on Flavobacterium growth and zeaxanthin biosynthesis. J. Indus. Microbiol. Biotechnol. 23, 697-700.
An, G. H., 1996. Characterization of yellow mutants isolated from the red yeast Phaffia rhodozyma. J. Microbiol. Biotechnol. 6, 110-115.
Bayne. C. K., Rubin, I. B., 1986. Optimization experiments In: Bayne. C. K., Rubin, I. B., (Eds), Practical experiments designs and optimization method for chemists. VCH publishers, Florida, pp. 105-144.
Ben-Amotz, A., 1998. Case study: Dunaliella algal-derived β-carotene. Phytochem. 113-128.
Ben-Amotz, A., 1999 Dunaliella β-carotene: from science to commerce. In: Cell. Origin Life Extreme Habitats, 1(Enigmatic Microorganisms and Life in Extreme Environments), pp: 401-410. Kluwer Academic Publishers.
Bhosale, P., Ermakov, I. V., Ermakova, M. R., Gellermann, W., Bernstein, P. S. 2003. Resonant Raman quantification of zeaxanthin production from Flavobacterium multivorum. Biotechnol. Lett. 25, 1007-1011.
Bhosale, P. B., Gadre, R. V. 2001. Production of β-carotene by a mutant of Rhodotorula glutinis. Appl. Microbiol. Biotechnol. 55, 423-427.
Box, G. P., Hunter, W. G. and Hunter, J. S. 1978. Factorial designs at two levels. In Statistics for Experiments: an Introduction to Design, Data Analysis and Model Building, 3 pp. 306-351. New York: John Wiley and Sons Inc.
Davies, L., 1993. The optimization of processes and products. In Efficiency in research, development and production: The statistical design and analysis of chemical experiments, pp. 124-127. Cambridge. Royal Society of Chemistry.
Ducrey Sanpietro, L. M., Kula, M. R. 1998. Studies of astaxanthin biosynthesis in Xanthophyllomyces dendrorhous (Phaffla rhodozyma). Effect of inhibitors and low temperature. Yeast. 14, 1007-1016.
Edge, R., McGarvey, D. J., Truscott, T. G. 1997. The carotenoids as antioxidants—a review. J. Photochem. Photobiol. B., 41, 189-200.
Fontana, J. D., Czeczuga, B., Bonfim, T. M. B., Chociai, M. B., Oliveira, B. H., Guimaraes, M. F., Baron, M., 1996. Bioproduction of carotenoids: the comparative use of raw sugarcane juice and depolymerized bagasse by Phaffia rhodozyma. Biores. Technol. 58, 121-125.
Girard, P., Falconnier, B., Bricout, J., Vladesco, B. 1994. β-Carotene producing mutants of Phafia rhodozyma. Appl. Microbiol. Biotechnol., 41, 183-191.
Gordon, H. T., Bauemfeind, J. C. 1982. Carotenoids as food colorants. Crit. Rev. Food Sci. Nutr. 18, 59-97.
Holick, C. N., Michaud, D. S., Stolzenberg-Solomon, R., Mayne, S. T., Pietinen, P., Taylor, P. R., Virtamo, J., Albanes, D. 2002. Dietary carotenoids, serum β-carotene, and retinol and risk of lung cancer in the α-tocopherol, α-carotene cohort study. Am. J. Epidemiol. 156, 536-547.
Johnson, E. A., Schroeder, W. A. 1996. Microbial carotenoids. Advan. Biochem. Eng. Biotechnol. 53, 119-178.
Kim, S. W., Seo, W. T., Park, Y. H. 1996. Increased β-carotene synthesis in Blakeslea trispora under strong alkaline culture condition Biotechnol. Lett. 18, 1287-1290.
Masetto, A., Flores-Cotera, L. B., Diaz, C., Langley, E., Sanchez, S. 2001. Application of a complete factorial design for the production of zeaxanthin by Flavobacterium sp. J. Biosci. Bioeng. 92, 55-58.
McDermott, J. C. B., Britton, G., Goodwin, T. W. 1973. Effect of Inhibitors on zeaxanthin synthesis in a Flavobacterium. J. Gen.l Microbiol. 77, 161-171.
Moeller, S. M., Jacques, P. F., Blumberg, J. B. 2000. The potential role of dietary xanthophylls in cataract and age-related macular degeneration. J. Am. Col. Nutr. 5, 522-527.
Montgomery, D. C. 1997. Chapter 14 Response surface methods and other approaches to process optimization. In: Montgomery, D. C. (Ed) Design and analysis of experiments-4^{th} Edition. John Wiley & Sons, Inc, NewYork, pp. 575-651.
Nelis, H. J., DeLeenheer, A. P. 1991. Microbial sources of carotenoid pigments used in food and feeds. J. App. Bacteriol. 70, 181-191.
Nishino, H., Murakosh, M., Takemura, M., Kuchide, M., Kanazawa, M., Mou X. Y. Wada, S., Masuda, M., Ohsaka, Y., Yogosawa, S., Satomi, Y., Jinno, K. 2002. Carotenoids in cancer chemoprevention. Cancer Metastasis Review. 21, 257-264.
Pasamontes, L., Hug, D., Tessier, M., Hohmann, H. P., Schierle, J., van Loon, A. P. 1997. Isolation and characterization of the carotenoid biosynthesis genes of Flavobacterium sp. strain R1534. Gene. 185, 35-41.
Ramirez, J., Gutierrez, H., Gschaedler, A. (2001) Optimization of astaxanthin production by Phaffia rhodozyma through factorial design and response surface methodology. J. Biotechnol. 88,259-268.
Seon-Won, K., Lee, I. Y., Jeong, J. C., Lee, J. H., Park, Y. H. 1999. Control of both foam and dissolved oxygen in the presence of a surfactant for production of β-carotene in Blakeslea trispora. J. Microbiol. Biotechnol. 9, 548-553.
Vazquez, M., Martin, A. M. 1998. Optimization of Phaffia rhodozyma continuous culture through response surface methodology. Biotechnol. Bioeng. 57,314-320.
   Each of the contents of the entirety of which are incorporated herein by this reference.

1. A process for producing a carotenoid in a bacterium, the process comprising:
supplementing a bacterial growth medium with a means for enhancing the production of the carotenoid from the bacterium; and
growing the bacterium in the bacterial growth medium.

2. The process of claim 1, further comprising isolating or purifying the carotenoid from the bacterial growth medium or the bacterium.

3. The process of claim 1, wherein the means for enhancing the production of the carotenoid is selected from the group consisting of calcium chloride, lithium chloride, magnesium chloride, magnesium sulfate, sodium carbonate, sodium chloride, sodium phosphate, ammonium chloride, ammonium phosphate, urea, isocitric acid, malic acid, α-ketoglutarate, citric acid and combinations of any thereof.

4. The process of claim 1, wherein the carotenoid is selected from the group consisting of β-carotene, β-cryptoxanthin, zeaxanthin, and combinations of any thereof.

5. The process of claim 1, wherein the means for enhancing the production of the carotenoid is present in the bacterial growth medium in a concentration of between about 100 and 400 mg/100 ml.

6. The process of claim 1, wherein the bacterium comprises a Flavobacterium species.

7. The process of claim 1, wherein the bacterium comprises Flavobacterium multivorum.

8. The process of claim 1, wherein the bacterial growth medium comprises glucose, yeast extract, peptone, and combinations of any thereof.

9. The process of claim 2, further comprising admixing the carotenoid with a pharmaceutical, a neutraceutical, a food stuff, a cosmetic or an animal feed.

10. A method for modulating carotenoid production in a bacterium, the method comprising:
supplementing a bacterial growth medium with a salt or a Krebs cycle intermediate, wherein the salt or the Krebs cycle intermediate in the supplemented bacterial growth medium is capable of attenuating the production of the carotenoid in the bacterium as compared to growth of the bacterium in the bacterial growth medium lacking the salt of the Krebs cycle intermediate; and
growing the bacterium in the supplemented bacterial growth medium.

11. The method according to claim 10, further comprising isolating or purifying the carotenoid from the supplemented bacterial growth medium or the bacterium.

12. The method according to claim 10, wherein the salt is selected from the group consisting of calcium chloride, lithium chloride, magnesium chloride, magnesium sulfate, sodium carbonate, sodium chloride, sodium phosphate, ammonium chloride, ammonium phosphate, urea and combinations of any thereof.

13. The method according to claim 10, wherein the carotenoid is selected from the group consisting of β-carotene, β-cryptoxanthin, zeaxanthin, and combinations of any thereof.

14. The method according to claim 10, wherein the Krebs cycle intermediate is selected from the group consisting of isocitric acid, malic acid, α-ketoglutarate, citric acid and combinations of any thereof.

15. The method according to claim 10, wherein bacterium comprises a Flavobacterium species.

16. The method according to claim 10, wherein the bacterium comprises Flavobacterium multivorum.

17. The method according to claim 10, wherein the bacterial growth medium comprises glucose, yeast extract peptone, and combinations of any thereof.

18. The method according to claim 11, further comprising admixing the carotenoid with a pharmaceutical, a nuetraceutical, a food stuff, a cosmetic or an animal feed.

19. A fermentor for growing bacteria, comprising:
an enhanced bacterial growth medium comprising at least one of glucose, yeast extract, and peptone;
means for enhancing the production of a carotenoid in a bacterium present at a concentration of between about 100 and 400 mg/100 ml; and
a culture of Flavobacterium multivorum.

20. The enhanced bacterial growth medium of claim 19, wherein the means for enhancing the production of the carotenoid in the bacterium is selected from the group of calcium chloride, lithium chloride, magnesium chloride, magnesium sulfate, sodium carbonate, sodium chloride, sodium phosphate, ammonium chloride, ammonium phosphate, urea, isocitric acid, malic acid, α-ketoglutarate and combinations of any thereof.
